(19) United States
(12) Patent Application Publication
(10) Pub. No.: US 2009/0203149 A1
(43) Pub. Date: Aug. 13, 2009

(54) ENHANCED METHODS FOR GAS AND/OR VAPOR PHASE ANALYSIS OF BIOLOGICAL ASSAYS

(75) Inventor: Andrew David Pris, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, Schenectady, NY (US)

(21) Appl. No.: 12/030,482

(22) Filed: Feb. 13, 2008

(51) Int. Cl. G01N 33/566

(52) U.S. Cl. 436/501

(57) ABSTRACT

Processes for improved efficiencies as it relates to the analysis of small molecules whose concentration in the analysis solution is dependent upon the concentration of a target as determined through a liquid phase biological assays with vapor and/or gas phase analysis are disclosed. The process generally includes the competitive or non-competitive binding of target substances onto carrier particles functioning as substrates in the biological assay. Employing the carrier particles as substrates provides increased surface area for the reaction to occur; increased ease of washing steps; and allows for concentration of the increased surface area into a smaller reaction volume prior to introduction into the vapor and/or gas phase spectrometer such as an ion mobility spectrometer.

### BACKGROUND

The present disclosure generally relates to enhanced methods for completing a biological assay for analysis by gas and vapor phase analytical methods, and more particularly, to the use of carrier particles as a support in an assay to enhance the gas and vapor phase analysis.

Gas and vapor phase analytical methods such as ion mobility spectrometry (IMS) can be used to detect and identify low concentrations of explosives, drugs, chemical weapons, and other chemicals of interest. This is accomplished through the IMS operation as exemplified in U.S. Pat. No. 3,699,333, U.S. Pat. No. 5,027,643, U.S. Pat. No. 5,491,337, and U.S. Pat. No. 6,690,005.

Efforts have been completed in the past to configure a biochemical test, or assay, such that a gas or vapor phase analytical device could analyze the assay and be capable of determining the presence and/or concentration of a designated target. In these biological assays through standard competitive or non-competitive assay processes, a labeled target binding moiety is present where either the label itself can be transferred to the gas phase or used to convert another molecule(s) into a product that can be transferred to the gas phase where they are analyzed by the gas phase analytical device for quantitative or qualitative information about the assayed target. Regina et al. (WO 88/06732) has shown the former method of directly analyzing the label that has been transferred to the gas phase. The latter method of employing a label that converts other molecule(s) into gas phase species has an advantage in trace biological analysis since a single label is used to convert numerous molecules into a product that is readily transferred to the gas phase. This effectively amplifies the presence of the target and assists in its detection. This second approach has also been shown in several different embodiments, all of which have limited detection capabilities due to the assay methods employed.

A specific embodiment of this second approach is the non-competitive enzyme linked immunosorbant assay (ELISA) where the target is captured on a solid support and specifically labeled with a biological recognition ligand (e.g., antibody) that is modified with an enzyme. After sufficient washing, the support is exposed to a solution that contains numerous precursor molecules that the enzyme repetitively converts into a form that is detectable by gas phase analytical devices. In an ELISA, the amount of the detectable molecule created is directly related to the concentration of the target present. In a related ELISA technique, the competitive format, the enzyme label is displaced from the support or must compete with target to bind to the support, resulting in both cases with less detectable molecule being created as the target concentration increases. Thus the value provided by the gas phase analytical device can still be used to relate to the amount of target present in the sample. Exemplary references in describing these standard assay format and terminology are contained in E. P. Diamandis, & T. K. Christopoulos (Immunoassay; Academic Press: 1996); S. S. Deshpande (Enzyme Immunoassays: From Concept to Product Development; Springer: 1996); and J. R. Crowther (The ELISA Guidebook (Methods in Molecular Biology); Humana: 1996).

Specifically, Diamond et al. (U.S. Pat. No. 4,629,689), Snyder et al. (Journal of Microbiological Methods. 27 (1996) 81-88 & U.S. Statutory Invention Registration H1563, Jul. 2, 1996), and Eiceman (Field Analytical Chemistry and Technology. 1(4):213-226, 1997) all show an ELISA that is analyzed with a gas phase analytical device. However, in each of these exemplary examples, the method by which the ELISA is run limits the simplicity, efficiency, and analytical utility of the overall biological detection process.

Diamond et al. attempts to improve this method through concentrating the gas phase species after volatilization. Eiceman et al. attempts to improve, not through analyzing the headspace, but rather by taking a portion of the un-concentrated reaction volume above a bulk support and analyzing it from a filter paper strips. Snyder et al. attempts to improve the known methodology by reducing the enzymatic reaction volumes through crude and inefficient methods such as “paddles/tissues” combinations.

In all cases, it would be extremely advantageous to employ a more global approach of concentrating the converter (or enzyme) prior to production of the detected molecule. It is well known that in the enzymatic and other catalytic conversion reactions, it is highly desirable to increase the concentration of the catalyst or enzyme within the reaction solution. This not only increases the reaction rates for the conversion process, thus allowing for a greater change in the amount of detectable molecules in a set period of time, but also accomplishes this in a smaller liquid volume. As the amount of liquid that is allowed to be introduced to gas phase analysis devices is limited, by completing the conversion in a decreased liquid volume, a larger fraction of the reaction solution can be sampled, thus delivering a larger fraction of the detectable molecule to the analysis device, with a concurrently decreased amount of liquid matrix that usually interferes or decreased the efficiency of small molecule vaporization and/or analysis.

A method for accomplishing this concentration of the converter prior to production of the gas phase molecule can be found in a separate body of science, where a plurality of target assay investigations have employed dispersed binding particles that possess micrometer or nanometer size and specific recognition capabilities (e.g., modified with antibody labels). It has been shown that these dispersed binding particles improve the efficiency and performance of the biological assay through increasing the surface area for the assay as well as allowing for increased target recognition kinetics over solid substrates or non-dispersed support beads. Moreover, the addition of specific physical and chemical properties (i.e., electrostatic, density, magnetic, refractive index, optical) to the micrometer or nanometer sized dispersed binding particle allows for efficient separation, and more importantly to this application, concentration of the target species. An exemplary example of dispersed binding particles for assays is found in U.S. Pat. No. 4,628,037.

However, combination of these two bodies of work (i.e., gas phase analysis of a bio-recognition scheme and employment of micrometer or nanometer sized dispersed binding particles) to increase binding kinetics, ease of separation, and label concentration, has not to date been specifically shown, described, or referred to.

Accordingly a need exists for a method that improves how an assay that is to be analyzed by a gas or vapor phase analysis device is performed to increase the efficiency of the binding recognition, increase the efficiency of the assay label conversion reaction, increase the fraction of assay components delivered to the analysis device and decrease the assay sample volume delivered to the analysis device.

### BRIEF SUMMARY

Disclosed herein are processes for gas and/or vapor analysis of biological assays. In one embodiment, a process for determining a target in a sample, the process comprises conducting an assay, wherein the assay comprises a dispersed carrier particle bound to a first target binder with a known target selectivity, a second target binder bound to a converter moiety, and a test sample that may contain a target in a first aqueous liquid; wherein the converter moiety selectively binds to the target, that is selectively bound to the carrier particle, in an amount dependent on a concentration of the target in the test sample; concentrating and separating the carrier particles from the first aqueous liquid; replacing the first aqueous liquid with a second aqueous liquid at a volume fraction of the first aqueous liquid volume and dispersing the carrier particles therein; applying a substrate to the dispersed carrier particles in the second aqueous liquid and converting the substrate with the converter moiety to form a product; and detecting the change in the substrate and/or product with vapor and/or gas phase analytical techniques.

In another embodiment, a process for analysis of detectable products produced in liquid phase biological assays with vapor and/or gas phase analysis comprises dispersing magnetic carrier particles modified with a first selective target binder and a converter moiety modified with a second selective target binder into an assay solution, wherein the magnetic carrier particles have an average particle size of 5 nanometers to 100 microns, wherein the converter moiety is an enzyme, and wherein the assay solution comprises a sample that is being examined for a target; creating a complex that consists of at least one magnetic particle, at least one of the targets, and at least one enzyme linked through the second selective target binder if the target is present; applying a magnetic field and concentrating the complexed and uncomplexed magnetic carrier particles from the uncomplexed enzymes and assay solution; re-dispersing the concentrated magnetic carrier particles into a second solution at a volume fraction of the first aqueous liquid volume, wherein the second solution contains a substrate for reaction with the complexed enzyme to produce a detectable product; detecting the detectable product by inserting an aliquot of the reaction solution into a gas and/or vapor phase analyzer, wherein the reaction solution contains the detectable product, un-reacted substrate, complexed magnetic carrier particles, and uncomplexed magnetic carrier particles, and wherein the substrate by itself is not detectable by the gas and/or vapor phase analyzer.

In still another embodiment, a process for analysis of detectable products produced in liquid phase biological assays with vapor and/or gas phase analysis comprises dispersing magnetic carrier particles that are modified with a first selective target binder and a converter moiety modified with a second selective target binder into an assay solution, wherein the magnetic carrier particles have an average particle size of 5 nanometers to 100 microns and the converter moiety is an enzyme, wherein the assay solution comprises a sample that is being examined for the target; creating a complex consists of at least one magnetic particle, at least one target, and at least one enzyme as linked through the respective target binders if the target is present; applying a magnetic field and concentrating the complexed and uncomplexed magnetic carrier particles from the un-complexed enzymes and assay solution; re-dispersing the concentrated magnetic carrier particles into a reaction solution wherein the solution contains a detectable substrate for reaction with the complexed enzyme to produce a non-detectable product; and detecting the detectable substrate by inserting an aliquot of the reaction solution into gas and/or vapor phase analyzer, wherein the reaction solution contains product, un-reacted substrate, complexed magnetic carrier particles, and uncomplexed magnetic carrier particles, wherein the product by itself is not detectable by the gas and/or vapor phase analyzer.

These and other features and advantages of the embodiments of the invention will be more fully understood from the following detailed description of the invention taken together with the accompanying drawings. It is noted that the scope of the claims is defined by the recitations therein and not by the specific discussion of features and advantages set forth in the present description.

### BRIEF DESCRIPTION OF THE DRAWINGS

FIG. 1 schematically illustrates one embodiment where a selective target binder (e.g., antibody) modified dispersed carrier particle, an enzyme that is attached to a target binder (e.g., antibody), and a sample that contains a target, upon interaction create a complexed carrier particle, which after concentration and washing, creates a detectable product from an undetectable substrate.

FIG. 2 schematically illustrates one embodiment where a selective target binder (e.g., antibody) modified dispersed carrier particle, an enzyme that is attached to a target binder (e.g., antibody), and a sample that does not contain a target upon interaction do not create complexed carrier particle, which after concentration and washing, does not create any detectable product from an undetectable substrate.

FIG. 3 schematically illustrates IMS response for a sample containing o-nitropheiol-beta-D-galactopyranoside (ONPG) as well as various negative and positive controls.

FIG. 4 schematically illustrates IMS response for a sample containing p-nitrophenol phosphate (PPNP), as well as, various negative and positive control.

Skilled artisans will appreciate that elements in the figures are illustrated for simplicity and clarity and have not necessarily been drawn to scale.

### DETAILED DESCRIPTION OF EMBODIMENTS OF THE INVENTION

Disclosed herein is a process for improved efficiencies as it relates to the analysis of small molecules produced in liquid phase biological assays with vapor and/or gas phase analysis. By way of example, specific reference will be made to small molecules produced by a non-competitive ELISA that are analyzed using ion mobility spectrometry (IMS), also referred to herein as ion trap mobility spectrometry (ITMS). However, it should be appreciated by those skilled in the art that the process can be applied to any liquid phase assay that employs a recognition moiety with known target selectivity and that is operated in a competitive or non-competitive fashion, where the assay outcome results in a change in the amount of the detectable molecule present, either through production or consumption of the detectable molecule, that is dependent upon the presence or concentration of the target. Likewise, the small molecules adjusted by the liquid phase assay can be analyzed using other types of vapor or gas phase analysis including, but not limited to, spectroscopy, mass spectrometry, differential mobility spectrometry, gas chromatography, and other analytical methods that combine selective analysis compounds and mass, electronic, optical and/or thermal transduction and the like.

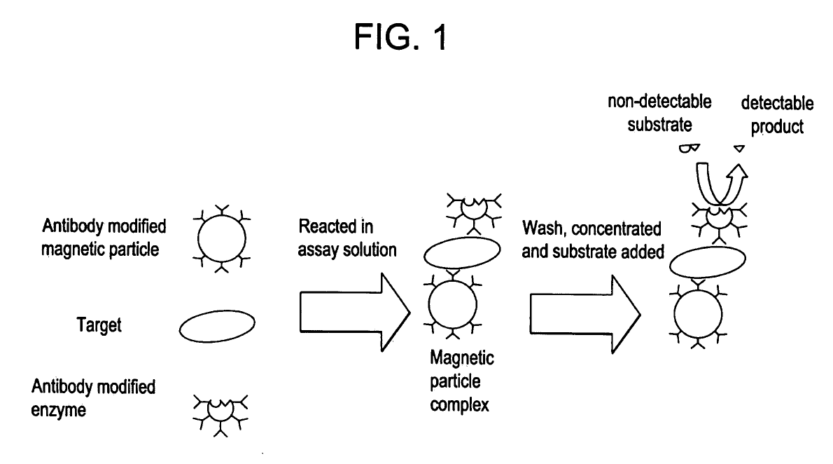

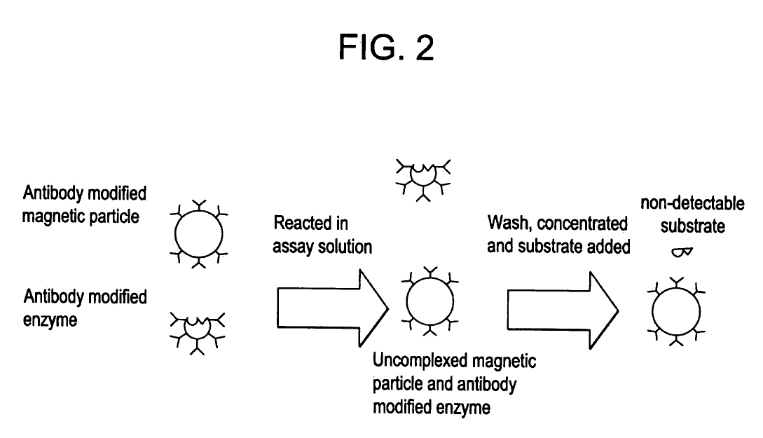

The process generally includes the binding of a target substance to both the target binder modified converter moiety as well as to the target binder modified carrier particles, wherein the carrier particles are micrometer or nanometer sized particles functioning as a capture phase in the biological assay. The carrier particles are configured to possess physical and/or chemical properties that allow for concentration of the labeling converter species. This forms the carrier particle complex as noted in a specific embodiment in FIG. 1. Importantly, if the target is not present, the carrier particle complex is not formed and there is no change in the amount of detectable compound as noted in a specific embodiment in FIG. 2. Non-specific binding sites may first be blocked with a suitable blocking agent, e.g., casein. In the non-competitive assay form, the assay labels the carrier particle captured target with a converter species that converts a molecule either into a form that is detectable, or not detectable, by a gas phase analysis. In the competitive assay form, the assay displaces converter species labels from the support or the converter species must compete with the target to bind to the support where the bound converter species converts a molecule either into a form that is detectable, or not detectable, by a gas phase analysis. Furthermore, employing the carrier particles as substrates provides increased surface area for the reaction to occur; increases the target binding kinetics; increases ease of washing steps; allows for concentration of the increased surface area into a smaller reaction volume for conversion of the non-detectable, or detectable, molecule; and, allows for increased ratio of assay components to conversation buffer matrix to be introduced to the gas phase analyzer. Because of the smaller reaction volume, the conversion of the precursor small molecule to the product small molecule will occur at a faster rate as it has been shown in the literature that enzyme catalysis is faster on particles than on solid substrates. Moreover, not only is the small molecule being created faster but the concentration will build up faster also due to the reaction occurring in a smaller volume which results in the small molecules reaching a critical concentration faster at which the small molecule can begin to build a partial pressure above the solution. The reduced reaction volume also allows for quicker vaporization of the entire solution and allows quicker liberation of the detectable small molecules from the liquid phase to the vapor phase. In addition, the reduced reaction volume increase the ratio between assay components to support buffer, which equates to less reaction volume components to be in the vapor phase, thus reducing background signal/noise.

Use of the dispersed carrier particles in the liquid phase assay process will affect the end user by improving the detection limit and sensitivity (through the increased surface area and more readily releasing the product into the vapor and/or gas phase to be detected as discussed above), will decrease the number of wash steps as well as decrease the overall number of assay steps; and it will decrease the amount of time to run the assay. The assay biorecognition kinetics are faster on the dispersed carrier particles as opposed to prior art supports, the small molecule conversion kinetics are faster, and the detectable small molecule will be released in greater amounts into the vapor and/or gas phase faster.

The carrier particles are dispersed into the assay solution during the assay. The carrier particles are selected such that the particles can be rapidly separated from the assay solution via optical, electrical, magnetic, gravitational or pressure forces. In one embodiment, the carrier particles are magnetic, which can be readily separated and/or concentrated from the assay solution by application of a magnetic field.

In the non-competitive assay format, preparation of assay samples generally includes mixing a target of interest, e.g., an antigen, with the target binder modified dispersed carrier particles, and a target binder with covalently bonded converter moiety in a suitable liquid, typically aqueous based to form a complexed carrier particle, as shown in one embodiment in FIG. 1. Alternatively, in the competitive assay format, the preparation of assay samples is similar to the non-competitive assay except the target binder modified converter moiety is either displaced by the target from the complex or competes with the target to bind to the carrier particle. Thus, the amount of converter moiety present upon the carrier particle is negatively correlated with the amount of target present. In both assay formats, the complexed and uncomplexed carrier particles are then separated from the supernatant. For example, as shown in FIG. 1, for magnetic particles, the particles are placed in a magnetic particle, concentrator for a period of time effective for the magnetic particles to collect along the sidewall, which is typically on the order of a few minutes to about 30 minutes, and the magnetic field within the concentrator is then discontinued and the supernatant removed. In this manner, separation times relative to prior art processes are substantially reduced. The carrier particles, once separated, are then washed in a suitable aqueous buffer one or more times. The concentrated particles are then dispersed with an enzymatic substrate to produce a detectable product if a target is present, thereby allowing for the formation of a complexed carrier particle. If the target is not present, the complexed carrier particle is not formed and there is no enzyme present in the second solution, thus no detectable product is created, as shown in FIG. 2. In all cases, an aliquot of the sample including the complexed particles, uncomplexed particles, unreacted substrate and the detectable product are then introduced into the vapor phase spectrometer, e.g., IMS. In the case of IMS, drift times are first collected for the respective detectable product. The sample is heated and the detectable product quantified. It should be noted that in the examples given above, a secondary antibody is not needed if the capture antibody incubated onto the carrier particles is conjugated to an enzyme. However, use of a secondary antibody conjugate avoids the expense of creating enzyme linked antibodies for every antigen one might want to detect.

The carrier particles are not intended to be limited to any particular type, although different types may provide different benefits. The carrier particles can be magnetic and separated from the assay during processing by means of an applied magnetic field; be electrically charged and separated from the assay by application of an electric field; have a particular refractive index in relation to the assay solution such that photons can be used to provide an optical force for separation, e.g., optical traps, optical tweezers, and the like or may have a relatively high density such that gravitational or centrifugal forces can be used concentrate the particles. The separation process will allow for concentration of the assay converter moiety and improve the aspects of the conversion process and of the interface to the gas phase analysis device. In applications where the density of the carrier particles is utilized, the density is in a range of about 0.1 mg/mL to about 1 mg/mL.

The carrier particles may be in any suitable form without any limitation as to the size and/or shape of the particles. In one embodiment, the particles are substantially spherical. For example, spherical particles with a diameter between about 5 nanometers and 100 microns may be used. In other embodiments, spherical particles with an average diameter between about 50 nanometers and 5 micron may be used. The modality of the particle size distribution can be unimodal, bimodal or multimodal. The size of the particles can influence a number of parameters. For example, if smaller particles are used, the maximum achievable array density is correspondingly greater. However, the larger surface area of a particle with a greater diameter allows the attachment of more targets per particle, resulting in a lower detection limit and greater analytical sensitivity and potentially greater signal intensity for each particle.

In one embodiment, the particles may comprise any appropriate magnetic material, e.g., iron (Fe), cobalt (Co), or nickel-iron alloys. As used herein, the term magnetic material includes paramagnetic materials. The particles may comprise nonmagnetic materials such as polystyrene in which magnetic subparticles (e.g., Fe₃O₄ particles) are embedded. Such particles may, for example, be dispersed throughout the nonmagnetic material or may form a core or shell below the surface of the nonmagnetic material. For biological applications, preferably at least the surface of the particle is made of a biocompatible material. Nonmagnetic biocompatible materials that may be used to coat the surface of a non-biocompatible material such as iron include polymeric materials such as polystyrene, latex, and numerous other materials well known in the art.

In certain embodiments, paramagnetic particles are used. Paramagnetic materials magnetize only when an external magnetic field is present, and thus paramagnetic particles exhibit minimal clumping. Biocompatible paramagnetic particles are available from a number of manufacturers (e.g., Dynal, Bangs Labs, Spherotech). Such particles are widely used for a variety of biological applications, and protocols for coupling biological molecules such as nucleic acids and proteins are well established. In addition, paramagnetic particles that are pre-conjugated with various binding ligands are available.

Superparamagnetic particles have a proven record of more than 15 years in commercial use. Such particles are manufactured by dispersing ferrite crystals throughout a polystyrene particle during its polymerization. The crystals are ferromagnetic, but because of their nanoscale size they behave not ferromagnetically but paramagnetically (the phenomenon has been termed superparamagnetism). It is believed that the orientational crystals are so small that they are randomized by thermal effects at room temperature. An array of such particles has essentially no remanence; it magnetizes substantially linearly in an applied magnetic field, losing essentially all magnetism when the external field is removed. This feature results in minimal clumping. The particles may be encapsulated for efficacy when used with enzymes (e.g., to avoid contact with iron-containing molecules) and the surface is easily modified to covalently attach biomolecules such as nucleic acids or proteins or small organic molecules.

A particle may include a detectable material such as a dye, a colorant, a hybridization tag or have a specific refractive index so that the particle may be detected on the array and identified among other particles as well as the assay solution. The detectable material can be incorporated within the particle, can be present on the surface, and/or can be linked to the particle. A particular detectable material or combination thereof can correspond to a particular probe that is attached to the particle, so that identification of the detectable material will also identify the probe. In certain embodiments, a particular detectable material can correspond to a particular target, so that identification of the detectable material will also identify a target that interacts with the probe.

The range of commercially available particles (both magnetic and nonmagnetic) is vast. Particles made of many different materials and sizes are available. Particles incorporating various molecules such as fluorescent dyes, particles conjugated with various moieties or having surfaces modified to facilitate such conjugation are available.

Suitable targets for use in the liquid phase assay include living targets and non-living targets. Examples of targets include, but are not limited to, prokaryotic cells, eukaryotic cells, bacteria, viruses, proteins, polypeptides, toxins, liposomes, particles, ligands, amino acids, nucleic acids, hormones, pharmaceuticals, toxic industrial chemicals, toxic industrial materials, and other small molecules either individually or in any combinations thereof. The target includes extracts of the above living or non-living targets.

Examples of prokaryotic cells include, but are not limited to, bacteria also include extracts thereof. Examples of eukaryotic cells include, but are not limited to, yeast cells, animal cells and tissues. Examples of toxins include, but are not limited to, anthrax. Examples of particles include, but are not limited to, latex, polystyrene, silica and plastic.

The term “peptide” refers to oligomers or polymers of any length wherein the constituent monomers are alpha amino acids linked through amide bonds, and encompasses amino acid dimers as well as polypeptides, peptide fragments, peptide analogs, naturally occurring proteins, mutated, variant or chemically modified proteins, fusion proteins, and the like. The amino acids of the peptide molecules may be any of the twenty conventional amino acids, stereoisomers (e.g., D-amino acids) of the conventional amino acids, structural variants of the conventional amino acids, e.g., iso-valine, or non-naturally occurring amino acids such as α,α-disubstituted amino acids, N-alkyl amino acids, β-alanine, naphthylalanine, 3-pyridylalanine, 4-hydroxyproline, O-phosphoserine, N-acetylserine, N-formylmethionine, 3-methylhistidine, 5-hydroxylysine, and nor-leucine. In addition, the term “peptide” encompasses peptides with posttranslational modifications such as glycosylations, acetylations, phosphorylations, and the like.

The term “oligonucleotide” is used herein to include a polymeric form of nucleotides of any length, either ribonucleotides or deoxyribonucleotides. This term refers only to the primary structure of the molecule. Thus, the term includes triple-, double-, and single-stranded DNA, as well as triple-, double-, and single-stranded RNA. The term also includes modifications, such as by methylation and/or by capping, and unmodified forms of the oligonucleotide. More particularly, the term includes polydeoxyribonucleotides (containing 2-deoxy-D-ribose), polyribo-nucleotides (containing D-ribose), any other type of polynucleotide which is an N-glycoside or C-glycoside of a purine or pyrimidine base, and other polymers containing nonnucleotidic backbones, for example, polyamide (e.g., peptide nucleic acids (PNAs)) and polymorpholine (commercially available from the Anti-Virals, Inc., Corvallis, Oreg., as Neugene) polymers, and other synthetic sequence-specific nucleic acid polymers, providing that the polymers contain nucleobases in a configuration that allows for base pairing and base stacking, such as is found in DNA and RNA. There is no intended distinction in length between the terms “polynucleotide”, “oligonucleotide”, “nucleic acid” and “nucleic acid molecule”, and these terms refer only to the primary structure of the molecule. Thus, these terms include, for example, 3′-deoxy-2′,5′-DNA, oligodeoxyribonucleotide N3′P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps”, substitution of one or more of the naturally occurring nucleotides with an analog, internucleotide modifications such as, for, example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphospho-triesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. The term also includes other kinds of nucleic acids such as, but not limited to, locked nucleic acids (LNAs).

The terms “nucleoside” and “nucleotide” also include those moieties that contain not only the known purine and pyrimidine bases, but also other heterocyclic bases, which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or, are functionalized as ethers, amines, or the like. The term “nucleotidic unit” is intended to encompass nucleosides and nucleotides.

Furthermore, modifications to nucleotidic units include rearranging, appending, substituting for or otherwise altering functional groups on the purine or pyrimidine base that form hydrogen bonds to a respective complementary pyrimidine or purine. The resultant modified nucleotidic unit optionally may form a base pair with other such modified nucleotidic units but not with A, T, C, G or U. Basic sites may be incorporated which do not prevent the function of the polynucleotide. Some or all of the residues in the polynucleotide optionally can be modified in one or more ways.

The term “antibody” as used herein includes antibodies obtained from both polyclonal and monoclonal preparations, as well as hybrid (chimeric) antibody molecules; F(ab′)2 and F(ab) fragments; Fv molecules (noncovalent heterodimers); single-chain Fv molecules (sFv); dimeric and trimeric antibody fragment constructs; humanized antibody molecules; and any functional fragments obtained from such molecules, wherein such fragments retain specific-binding properties of the parent antibody molecule.

Binding of the targets to the particles is controlled by the surface chemistry of the particles. The surface chemistry of the particles may be used for binding target substances via non-specific interactions, such as, for example, electrostatic interactions, van der Waals interactions, dipole-dipole interactions, and/or hydrogen bonding interactions. Alternatively, the carrier particles can be chemically modified to include specific binding substances that interact selectively with the target substances. Representative examples of binding substances that can be used include, without limitation, antigens, antibodies, aptamers, polypeptides, peptides, nucleic acids, protein receptors, ligands, oligonucleotides, streptavidin, avidin, biotin, lectin, and the like.

Target-binding moieties may attach to the target, directly or indirectly. Examples of attaching include, but are not restricted to, electrostatically, chemically, and physically. Examples of target-binding moieties include, but are not limited to, antibodies, aptamers, polypeptides, peptides, nucleic acids, avidin, streptavidin, and derivatives of avidin and streptavidin, either individually or in any combination thereof.

Other non-limiting examples of target-binding moieties include, but are not limited to, proteins, peptides, polypeptides, glycoproteins, selected ligands, lipoproteins, phospholipids, oligonucleotides, or the like, e.g. enzymes, immune modulators, receptor proteins, antibodies and antibody fragments, which preferentially bind marker substances that are produced by or associated with the target site

Proteins are known that preferentially bind marker substances that are produced by or associated with lesions. For example, antibodies can be used against cancer-associated substances, as well as against any pathological lesion that shows an increased or unique antigenic marker, such as against substances associated with cardiovascular lesions, for example, vascular clots including thrombi and emboli, myocardial infarctions and other organ infarcts, and atherosclerotic plaques; inflammatory lesions; and infectious and parasitic agents.

Cancer states include carcinomas, melanomas, sarcomas, neuroblastomas, leukemias, lymphomas, gliomas, myelomas, and neural tumors. Infectious diseases include those caused by body invading microbes or parasites.

The protein substances useful as target-binding moieties include protein, peptide, polypeptide, glycoprotein, lipoprotein, or the like; e.g. hormones, lymphokines, growth factors, albumin, cytokines, enzymes, immune modulators, receptor proteins, antibodies and antibody fragments. The protein substances of particular interest are antibodies and antibody fragments. The terms “antibodies” and “antibody fragments” mean generally immunoglobulins or fragments thereof that specifically bind to antigens to form immune complexes.

The antibody may be a whole immunoglobulin of any class; e.g., IgG, IgM, IgA, IgD, IgE, chimeric or hybrid antibodies with dual or multiple antigen or epitope specificities. It can be a polyclonal antibody, particularly a humanized or an affinity-purified antibody from a human. It can be an antibody from an appropriate animal; e.g., a primate, goat, rabbit, mouse, or the like. If a paratope region is obtained from a non-human species, the target may be humanized to reduce immunogenicity of the non-human antibodies, for use in human diagnostic or therapeutic applications. Such a humanized antibody or fragment thereof is termed “chimeric.” For example, a chimeric antibody comprises non-human (such as murine) variable regions and human constant regions. A chimeric antibody fragment can comprise a variable binding sequence or complementarity-determining regions (“CDR”) derived from a non-human antibody within a human variable region framework domain. Monoclonal antibodies are also suitable because of their high specificities. Useful antibody fragments include F(ab′)₂, F(ab)₂, Fab′, Fab, Fv, and the like including hybrid fragments. Particular fragments are Fab′, F(ab′), Fab, and F(ab)₂. Also useful are any subfragments retaining the hypervariable, antigen-binding region of an immunoglobulin and having a size similar to or smaller than a Fab′ fragment. An antibody fragment can include genetically engineered and/or recombinant proteins, whether single-chain or multiple-chain, which incorporate an antigen-binding site and otherwise function in vivo as immobilized target-binding moieties in substantially the same way as natural immunoglobulin fragments. The fragments may also be produced by genetic engineering.

Examples of selective ligands include porphyrins, ethylene-diaminetetraacetic acid (EDTA), and zinc fingers. Selective ligand means a ligand selective for a particular target or targets.

Mixtures of antibodies and immunoglobulin classes can be used, as can hybrid antibodies. Multispecific, including bispecific and hybrid, antibodies and antibody fragments are sometimes desirable for detecting and treating lesions and include at least two different substantially monospecific antibodies or antibody fragments, wherein at least two of the antibodies or antibody fragments specifically bind to at least two different antigens produced or associated with the targeted lesion or at least two different epitopes or molecules of a marker substance produced or associated with the targeted lesion. Multispecific antibodies and antibody fragments with dual specificities can be prepared analogously to anti-tumor marker hybrids.

Suitable MAbs against microorganisms (bacteria, viruses, protozoa, other parasites) responsible for the majority of infections in humans may be used for in vitro diagnostic purposes. These antibodies, and newer MAbs, are also appropriate for use.

Proteins useful for detecting and/or treating cardiovascular lesions include fibrin-specific proteins; for example, fibrinogen, soluble fibrin, antifibrin antibodies and fragments, fragment E₁ (a 60 kDa fragment of human fibrin made by controlled plasmin digestion of crosslinked fibrin), plasmin (an enzyme in the blood responsible for the dissolution of fresh thrombi), plasminogen activators (e.g., urokinase, streptokinase and tissue plasminogen activator), heparin, and fibronectin (an adhesive plasma glycoprotein of 450 kDa) and platelet-directed proteins; for example, platelets, antiplatelet antibodies, and antibody fragments, anti-activated platelet antibodies, and anti-activated platelet factors.

In one embodiment, the target-binding moiety includes a MAb or a fragment thereof that recognizes and binds to a heptapeptide of the amino terminus of the β-chain of fibrin monomer. Fibrin monomers are produced when thrombin cleaves two pairs of small peptides from fibrinogen. Fibrin monomers spontaneously aggregate into an insoluble gel, which is further stabilized to produce blood clots.

The disclosure of various antigens or biomarkers that can be used to raise specific antibodies against them (and from which antibodies fragments may be prepared) serves only as an example, and is not to be construed in any way as a limitation of the invention.

The assay can be conducted with the described dispersed carrier particles in a competitive or a non-competitive fashion. It is well known to those skilled in the art that a non-competitive assay will result in more of the converter moiety to be present in the conversion reaction whereas a competitive assay will result in less of the conversion moiety to be present in the conversion reaction. As a result of the assay being competitive or non-competitive, for any given converter moiety the change in the amount of detectable molecule due to increases in the concentration of the target species will be opposite and monitored accordingly.

The converter moiety is directly attached with a target binding moiety through, but not restricted to, electrostatic, chemical, and/or physical means. The converter moiety is responsible for either creating or suppressing the detectable molecule for the gas phase analysis. Non-limiting examples of these include inorganic, organic or biological catalyst that allow for redox, electronic or enzymatic conversion.

The converter moiety acts as an amplifier; even if only a few converter moieties linked to binding moieties remain bound, the converter moiety will convert many signal molecules. The present disclosure is not intended to be limited to any particular converter moiety and will generally depend on the detectable/non-detectable molecule; selection of which is well within the skill of those in the art.

The converter moiety may work to either create the detectable species from a detectable species from a non-detectable form (e.g., hydrolysis of a sugar group from the detectable species as with a galactosidase) or alternatively create a non-detectable form from the detectable form (e.g., creation of a radical group that polymerizes or combines two molecules of the detectable form as with a peroxidase). In each of the cases the gas phase analysis device will monitor the creation or reduction of the detectable species and relate that to either the presence (qualitative) or amount (quantitative) of the converter moiety present. It is also to be understood that a reaction scheme that is initiated by the action of the converter moiety that eventually affects the form of the detectable species is also included.

“Substrates” refers to the starting form of the molecule that the converter moiety changes into the “product” or the end form of the molecule after an action of the converter moiety. The substrate and product must be able to be differentiated by the gas phase analyzer. In a preferred embodiment, either the substrate or the product will not be detected by the gas phase analyzer while their other complement is detectable.

The gas phase ion spectrometer refers to any apparatus that detects gas phase ions. Gas phase ion spectrometers include an ion source that supplies gas phase ions. Gas phase ion spectrometers include, for example, mass spectrometers, ion mobility spectrometers, and total ion current measuring devices. In one embodiment, an IMS is used to detect and characterize then detectable product of the assay. The carrier particles and the substrate in the liquid phase are placed within the IMS and heated to a temperature from about 25° C. to about 600° C. depending on the detectable molecule, e.g., the product produced by the enzyme-substrate reaction where the substrate by itself is not detectable.

The following example illustrates the features of the disclosure and is not intended to limit the disclosure thereto. In the examples, an Itemiser³® ITMS instrument running software version 8.12 (GE Security, Bradenton, Fla.) or a VaporTracer²® ITMS instrument running software version 3.19 (GE Security, Bradenton, Fla.) was set in explosive mode with a default sampling time of 7 seconds, a desorber temperature of 220° C. and a detector temperature of 205° C. Prior to all experimental runs, the unit was calibrated according to the vendor specifications in dual mode using calibration traps (Part #M0001319). The systems were run with the semi-permeable membrane (Part #PA005007) in place and with the explosive dopant (methlyene chloride, Part #MP005810) present within the system.

### EXAMPLE

In the examples, the following chemicals and reagents were used. Sodium phosphate dibasic, 2-(N-Morpholino)ethanesulfonic acid (MES), glycine, Tris(hydroxymethyl)aminomethane hydrochloride (Tris), Tween-20, bovine serum albumin (BSA), TritonX-100, orthonitrophenol-beta-D-galactopyranoside (ONPG), paranitrophenol phosphate (PNPP), orthonitrophenol (ONP), paranitrophenol (PNP) were obtained and used as received from Sigma-Aldrich, Inc. (St. Louis, Mo.). Sodium chloride, sodium hydroxide, and hydrochloric acid (concentrated) were used as received from Fisher Scientific Inc. (Pittsburgh, Pa.). The following buffers were made with 18 MΩ Milli-Q water (Millipore, Billerica, Mass.) and brought to the proper pH with sodium hydroxide or hydrochloric acid; 10 mM phosphate, 137 mM NaCl, 0.1% (w/w) TritonX-100, 0.1% (w/w) sodium azide (pH 7.4); 20 mM Tris, 500 mM NaCl, 1% BSA, 0.1% Tween-20 (pH 7.6); 100 mM glycine, 125 mM NaCl, 1 mM MgCl, 1 mM ZnCl₂ (pH 10.0); 25 mM MES (pH 6.0). Dynabeads anti-E. coli 0157 were purchased and washed in the phosphate buffer for the ONPG assay or the Tris buffer for the PNPP assay according to vendor protocol and diluted to ˜1×10⁸ particle/mL (Invitrogen, Carlsbad, Calif.). Affinity purified goat anti-E. coli O157:H7, phosphatase-labeled affinity purified goat anti-E. coli O157:H7 (0.1 mg/ml), and E. coli O157:H7 positive control, were obtained from KPL (Gaithersburg, Md.) and rehydrated in 50% water: 50% glycerol and stored at 4° C. until required. Beta-galactosidase conjugated streptavidin was purchased and used as received (Rockland, Gilbertsville, Pa.). EZ-Link Sulfo-NHS-LC-LC-Biotin (Thermo-Pierce, Rockford, Ill.) was used according to vendor protocols in 25 mM MES to biotinylated the goat anti-E. coli antibody. Purification steps were completed with Microcon YM-50 spin filters (Millipore) with the phosphate buffer. A 4:1 molar ratio of biotinylated antibody to streptavidin-modified enzyme were combined and allowed to react for 4 hours at 4° C. to provide a final antibody concentration of ˜0.1 mg/mL in the phosphate buffer.

The following substrate and product solutions were created at a 1 mg/mL concentration: ONPG in phosphate buffer; ONP in phosphate buffer; PNPP in glycine buffer; PNP in glycine buffer.

ONPG Assay: The following were combined in a 600 microL polypropylene microcentrifuge tube (Fisher Scientific): 150 microL phosphate buffer, 10 microL goat anti-E. coli magnetic particles in phosphate buffer, 10 microL 1×10⁹ cells/mL E. coli positive control, 10 microliters of the 0.1 mg/mL goat anti-E. coli-biotin-streptavidin-galactosidase in phosphate buffer. These were briefly vortexed and allowed to rock on a rotating platform for 30 min at room temperature. At this point in time, the microcentrifuge tube was placed in a magnetic particle concentrator rack (Dynal) for 1 minute allowing the magnetic particles to collect along the side wall. The supernatant was removed and replaced with 300 microL of the phosphate buffer into which the particles were re-suspended via slight vortexing. This was repeated twice more and finally the particles were dispersed in 100 microL of the 1 mg/mL ONPG substrate in the phosphate buffer and heated at 37° C. for 30 min.

PNPP Assay: The following were combined in a 600 microL polypropylene microcentrifuge tube (Fisher Scientific): 150 microliters Tris buffer, 10 microliters goat anti-E. coli magnetic particles in Tris buffer, 10 microL 1×10⁹ cells/mL E. coli positive control, 10 microliters of the 0.1 mg/mL goat anti-E. coli-phosphatase in Tris buffer. These were briefly vortexed and allowed to rock on a rotating platform for 30 min at room temperature. At this point in time, the microcentrifuge tube was placed in a particle concentrator rack for 1 min allowing all of the magnetic particles to collect along the side wall. The supernatant was removed and replaced with 300 microliters of the Tris buffer into which the particles were re-suspended via slight vortexing. This was repeated twice more and finally the particles were dispersed in 100 microliters of the 1 mg/mL PNPP substrate in the glycine buffer and heated at 37° C. for 30 min.

Specifically, 10 microliters of a sample solution Was pipetted upon a woven “gold” sample trap (Part # M0001249, GE Security, Bradenton, Fla.) and immediately inserted into the “particulate” sampling port of the instrument. The system was then triggered to acquire the sample. Upon prompting by the instrument, the sample trap was removed and discarded. The collected data set consisted of 70 spectra, or plasmagrams, that were separated by 100 ms intervals, these were all saved and analyzed on a separate computer.

Prior to examining the galactosidase or phosphatase assay samples, the 1 mg/mL ONP in phosphate buffer and 1 mg/mL PNP in glycine buffer were examined in a similar manner to how the assay samples were examined to determine the respective drift times of the product molecules. Additionally, the 1 mg/mL ONPG in phosphate buffer and 1 mg/mL PNPP in glycine buffer were sampled to insure that these substrate molecules could not be detected by the IMS unit.

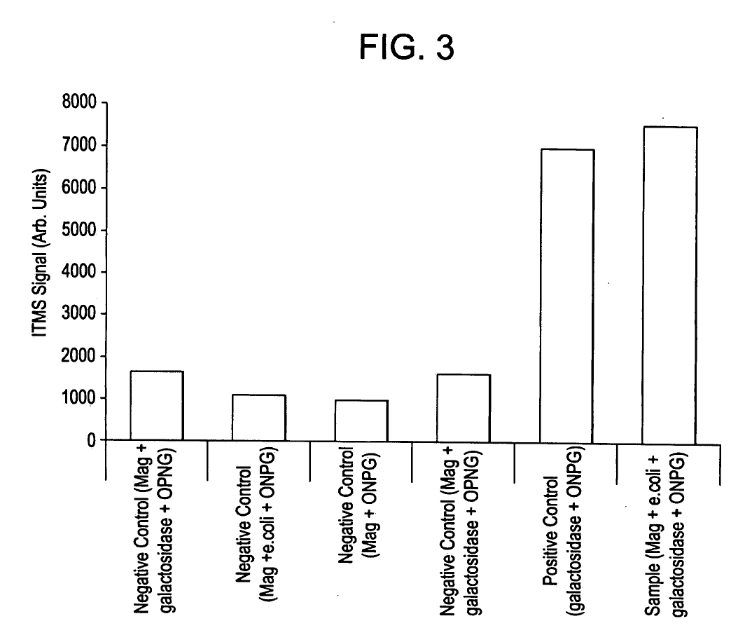

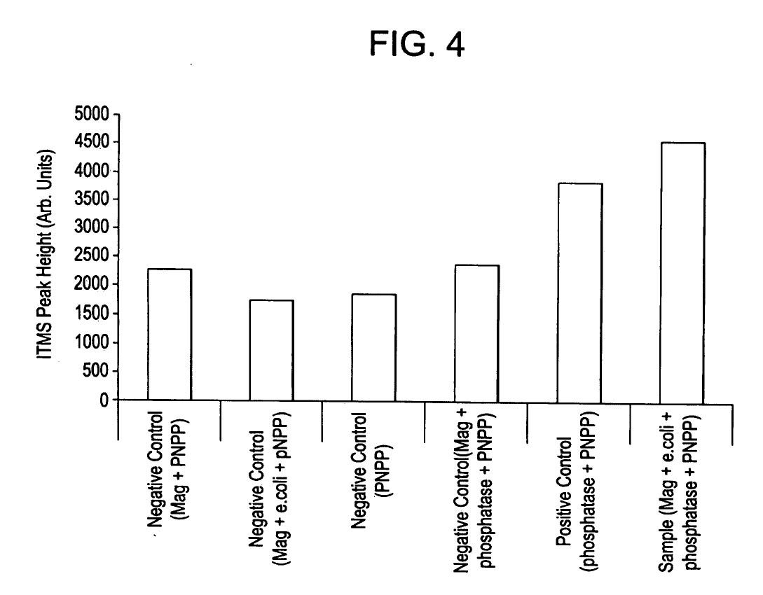

After analysis of the assay solutions by the IMS unit, the collected data was examined and the peak height of the spectra that was associated with the product molecule was collected. The peak height associated with the product drift time for various sample associated with the assay are displayed in FIGS. 3 and 4 to display the ability to use the magnetic particles in an assay with IMS analysis. FIGS. 3 and 4 also display several negative and positive controls.

Advantageously, the use of dispersed carrier particles as described herein allows for an increased amount of surface area to be present to create the product; it allows for concentration of the surface area into a small reaction volume which allows for the small molecule to be more readily released into the vapor and/or gas phase through normal development of partial pressure or through increased ease of vaporization. Moreover it decreases the number of overall steps in the assay process as well.

It will be understood that when an element is referred to as being “on” another element, it can be directly on the other element or intervening elements can be present there between. In contrast, when an element is referred to as being “disposed on” or “formed on” another element, the elements are understood to be in at least partial contact with each other, unless otherwise specified.

The terminology used herein is for the purpose of describing particular embodiments only and is not intended to be limiting of the invention. As used herein, the singular forms “a”, “an” and “the” are intended to include the plural forms as well, unless the context clearly indicates otherwise. The use of the terms “first”, “second”, and the like do not imply any particular order, but are included to identify individual elements. It will be further understood that the terms. “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which the embodiments of the invention belong. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

While embodiments of the invention have been described with reference to exemplary embodiments, it will be understood by those skilled in the art that various changes can be made and equivalents can be substituted for elements thereof without departing from the scope of the embodiments of the invention. In addition, many modifications can be made to adapt a particular situation or material to the teachings of embodiments of the invention without departing from the essential scope thereof. Therefore, it is intended that the embodiments of the invention not be limited to the particular embodiment disclosed as the best mode contemplated for carrying out this invention, but that the embodiments of the invention will include all embodiments falling within the scope of the appended claims.

1. A process for determining a target in a sample, the process comprising, in sequence:
conducting an assay, wherein the assay comprises a dispersed carrier particle bound to a first target binder with a known target selectivity, a second target binder bound to a converter moiety, and a test sample that may contain a target in a first aqueous liquid; wherein the converter moiety selectively binds to the target, that is selectively bound to the carrier particle, in an amount dependent on a concentration of the target in the test sample;
concentrating and separating the carrier particle from the first aqueous liquid;
replacing the first aqueous liquid with a second aqueous liquid at a volume fraction of the first aqueous liquid volume and dispersing the carrier particle therein;
applying a substrate to the dispersed carrier particle in the second aqueous liquid and converting the substrate with the converter moiety to form a product; and
detecting the change in the substrate and/or product with vapor and/or gas phase analytical techniques.

2. The process in claim 1, wherein the target increases association of the converter moiety with the carrier particle.

3. The process of claim 1, wherein the target decreases association of the converter moiety with the carrier particle

4. The process of claim 1, wherein the carrier particles are magnetic particles and said separating the carrier particles from the first liquid comprises applying a magnetic field.

5. The process of claim 1, wherein the carrier particles are electrically charged particles and said separating the carrier particles from the first liquid comprises applying an electric field.

6. The process of claim 1, wherein the carrier particles have a refractive index in relation to the first aqueous liquid that allow for optical forces to concentrate and separate the carrier particles from the first liquid.

7. The process of claim 1, wherein the carrier particles have an average particle size from about 5 nanometers to about 100 microns.

8. The process of claim 1, wherein said separating the carrier particles from the first aqueous liquid is for a period of time less than about 30 minutes.

9. The process in claim 1, wherein the product produced by conversion of the substrate by the converter moiety is a detectable product and the substrate is non-detectable.

10. The process of claim 9, wherein said detecting the change in the substrate and/or product with vapor and/or gas phase analytical techniques comprises inserting, an aliquot of the second aqueous liquid with the carrier particles and the detectable product into an ion mobility spectrometer.

11. The process in claim 1, wherein the product produced by conversion of the substrate by the converter moiety is a non-detectable product and the substrate is detectable.

12. The process of claim 11, wherein said detecting the change in the substrate and/or product with vapor and/or gas phase analytical techniques comprises inserting an aliquot of the second aqueous liquid with the carrier particles and the detectable substrate into an ion mobility spectrometer.

13. The process of claim 1, wherein the vapor and/or gas phase analytical technique produces an output for quantitatively or qualitatively determining a presence or an amount of the selected target in the sample.

14. The process of claim 1, wherein the target comprises an antibody or an antigen.

15. The process of claim 1, wherein the first and second target binders comprise at least one chemical moiety selected from a group consisting of antigens, antibodies, aptamers, polypeptides, peptides, nucleic acids, protein receptors, ligands, oligonucleotides, streptavidin, avidin, biotin, lectin, and the like.

16. The process of claim 1, wherein the first and second target binders are the same.

17. A process for analysis of detectable products produced in liquid phase biological assays with vapor and/or gas phase analysis, the process comprising:
dispersing magnetic carrier particles modified with a first selective target binder and a converter moiety modified with a second selective target binder into an assay solution, wherein the magnetic carrier particles have an average particle size of 5 nanometers to 100 microns, wherein the converter moiety is an enzyme, and wherein the assay solution comprises a sample that is being examined for a target;
creating a complex that consists of at least one magnetic carrier particle, at least one target, and at least one enzyme linked through the second selective target binder if the target is present;
applying a magnetic field and concentrating the complexed and uncomplexed magnetic carrier particles from the uncomplexed enzymes and assay solution;
re-dispersing the concentrated magnetic carrier particles into a second solution, wherein the second solution contains a substrate for reaction with the complexed enzyme to produce a detectable product; and
detecting the detectable product by inserting an aliquot of the reaction solution into a gas and/or vapor phase analyzer, wherein the reaction solution contains the detectable product, un-reacted substrate, complexed magnetic carrier particles, and uncomplexed magnetic carrier particles, and wherein the substrate by itself is not detectable by the gas and/or vapor phase analyzer.

18. The process of claim 17, comprising blocking non-specific binding sites prior to mixing with the sample that is being examined for the target.

19. The process of claim 17, wherein said creating a complex is competitive such that an amount of the complex present negatively correlates with an amount of the target present in the sample.

20. The process of claim 17, wherein said creating a complex is non-competitive such that an amount of the complex present positively correlates with an amount of the target present in the sample.

21. The process of claim 17, wherein the gas and/or vapor phase analyzer is an ion mobility spectrometer.

22. The process of claim 17, wherein the gas and/or vapor phase analyzer is an ion trap mobility spectrometer.

23. The process of claim 17, wherein the gas and/or vapor phase analyzer provides an output for qualitatively or quantitatively determining a presence or an amount of the selected target in the sample.

24. The process of claim 17, wherein the target comprises an antibody or an antigen.

25. The process of claim 17, wherein separating the carrier particles from the first aqueous liquid is for a period of time less than about 30 minutes.

26. The process of claim 17, wherein the selective target binders comprise at least one chemical moiety selected from a group consisting of antigens, antibodies, aptamers, polypeptides, peptides, nucleic acids, protein receptors, ligands, oligonucleotides, streptavidin, avidin, biotin, lectin, and the like.

27. A process for analysis of detectable products produced in liquid phase biological assays with vapor and/or gas phase analysis, the process comprising:
dispersing magnetic carrier particles that are modified with a first selective target binder and a converter moiety modified with a second selective target binder into an assay solution, wherein the magnetic carrier particles have an average particle size of 5 nanometers to 100 microns and the converter moiety is an enzyme, wherein the assay solution comprises a sample that is being examined for the target;
creating a complex consists of at least one magnetic particle, at least one target, and at least one enzyme as linked through the respective target binders if the target is present;
applying a magnetic field and concentrating the complexed and uncomplexed magnetic carrier particles from the un-complexed enzymes and assay solution;
re-dispersing the concentrated magnetic carrier particles into a reaction solution wherein the solution contains a detectable substrate for reaction with the complexed enzyme to produce a non-detectable product; and
detecting the detectable substrate by inserting an aliquot of the reaction solution into gas and/or vapor phase analyzer, wherein the reaction solution contains product, un-reacted substrate, complexed magnetic carrier particles, and uncomplexed magnetic carrier particles, wherein the product by itself is not detectable by the gas and/or vapor phase analyzer.

28. The process of claim 27, comprising blocking non-specific binding sites prior to mixing with the sample.

29. The process of claim 27, wherein said applying a magnetic field separates the magnetic carrier particles from the assay solution and discontinuing the magnetic field re-disperses the magnetic particles into the reaction solution.

30. The process of claim 27, wherein said creating a complex is competitive such that an amount of the complex present negatively correlates with an amount of the target present in the sample.

31. The process of claim 27, wherein said creating a complex is non-competitive such that an amount of the complex present positively correlates with an amount of target present in the sample.

32. The process of claim 27, wherein the gas and/or vapor phase analyzer is an ion mobility spectrometer.

33. The process of claim 27, wherein the gas and/or vapor phase analyzer is a ion trap mobility spectrometer.

34. The process of claim 27, wherein the gas and/or vapor phase analyzer provides an output for qualitatively or quantitatively determining a presence or an amount of the selected target in the sample.

35. The process of claim 27, wherein the target comprises an antibody or an antigen.

36. The process of claim 27, wherein said applying a magnetic field and said re-dispersing the carrier particles from the assay solution is for a time less than about 30 minutes

37. The process of claim 27, wherein the target binders comprise at least one chemical moiety selected from a group consisting of antigens, antibodies, aptamers, polypeptides, peptides, nucleic acids, protein receptors, ligands, oligonucleotides, streptavidin, avidin, biotin, lectin, and combinations thereof.
